(19) United States
(12) Patent Application Publication
(10) Pub. No.: US 2007/0100401 A1
(43) Pub. Date: May 3, 2007

(54) Compact laser device and method for hair removal

(76) Inventor: J.T. Lin, Oviedo, FL (US)

Correspondence Address: J.T. Lin, 4532 Old Carriage Trail, Oviedo, FL, 32765, US

(21) Appl. No.: 11/242,187

(22) Filed: Nov. 1, 2005

(51) Int. Cl. A61N 5/06; A61B 18/18

(52) U.S. Cl. 607/89; 606/9

(57) ABSTRACT

A device for hair removal includes the use of infrared laser having wavelength of about 0.7 to 1.1 microns, energy per pulse of about 0.5 to 5.0 J on the skin surface and operated at about 1.0 to 5.0 Hz. The treated area includes one or more than one of the following: the hair shaft, root, hair follicle, papilla, blood vessels feeding the papilla, or blood vessels in the papilla. The delivery means includes an optical fiber or fiber bundle which delivers said laser beam to said treated skin, where the optical fibers is further connected to a hand piece containing the laser unit and optics. The laser beam is generated from a laser unit consisting of about 1 to 5 diode arrays having the same wavelength at about 0.7 to 1.1 microns, or a combination of 2 to 3 different wavelengths selected from the ranges of about 700 to 760 nm, 780 to 820 nm, 900 to 930 nm, or 970 to 990 nm.

### BACKGROUND OF THE INVENTION

1. Field of the Invention

The present invention relates to laser device and method for hair removal. More particularly, it relates to systems of using a compact diode laser device at low power with multiple wavelength output for all skin type and hair color.

2. Prior Art

Different lasers have been disclosed for hair removal since the first low energy normal mode ruby (at 694 nm) and Nd:YAG (at 1064 nm) lasers were disclosed in 1967, U.S. Pat. No. 3,538,919. These prior arts include the earlier approaches using a pulsed, or Q-switched ruby laser disclosed in 1990 by Zaim in U.S. Pat. No. 5,059,192; high energy normal mode ruby laser disclosed in 1997 by Anderson et. al. in U.S. Pat. No. 5,595,568; and the more recent patents of Anderson et. al. (U.S. Pat. Nos. 5,735,884, 6,183,773), Tankovick (U.S. Pat. No. 5,752,948). Alexandrite laser (at 755 nm) was proposed in U.S. Pat. No. 5,879,346 (of Waldman et al); U.S. Pat. Nos. 5,871,479, 6,045,548 and 6,632,218 (of Furumoto et al). More recently, in 2003, U.S. Pat. No. 6,595,985 (of Tobinick), use of pulse groups with adjustable pulse width and delay time were disclosed for different skin types and hair color treatment. All of the above cited prior arts are using a single wavelength laser.

A recent patent of Stewart, U.S. Pat. No. 6,544,255, disclosed the use of dual-wavelength system, one at (900-950) nm and another one at its second-harmonic at (450-475) nm, used for large area (or transcutaneous) and for single hair (or intrafollicular) treatment, respectively. The proposed non-specified laser, however, is not yet technically available, except a low power diode laser which can not be frequency doubled to 450 to 470 nm. Therefore, the method proposed in this prior art is not technically practical, and no systems have been made based on this method.

The existing commercial lasers for hair removal are mainly limited to Alexandrite (at 755 nm) and diode laser (at about 810 nm) using a typical beam spot of 9 mm, power output about 50 to 100 W, energy per pulse of 10 to 40 J, and operated at about 0.5 to 2 Hz low repetition rate. These high-power systems are designed for large area, fast treatment. A typical system has a weight over 50 pounds and dimension over 15×15×20 inch. In addition, they all use a single fixed wavelength and non-focused laser which limits the laser penetration to a fixed depth and therefore it is not optimized for different skin types or hair color, although pulse duration of 5 to 400 microseconds was adjustable in these commercial systems. These high energy, high power systems also suffer from the risk of burning or damaging the skin epidermis, even step of cooling is included. Furthermore, the existing systems generally require 3 to 4 treatments because they can not produce sufficient temperature in those hair follicles which do not contain a hair shaft or have a deep roots (deeper than 5 mm).

Because of the disadvantages and limitations associated with both methods and devices in use today, a new method and system are needed for more convenient, low cost and, most importantly, more efficient for all skin types and hair color. Furthermore, there is a need of compact size, low-power laser device for personal or family uses, rather than the high-power, high-cost and bulky system which is limited for clinical or hospital use. The handheld compact laser based on optimal lens design may partially replace the traditional razor by offering the advantage of either long-term or permanent facial hair removal, rather than the daily shaving. In addition, a razor used in public barbershops suffers the risk of contracting AIDS which can be totally avoided when a laser is used in a non-contact mode.

### SUMMARY OF THE INVENTION

The preferred embodiments of the basic lasers of the present invention shall include a compact laser device using low power (0.5 to 15 W), low energy (0.5 to 5.0 J) and small circular spot (about 2 to 5 mm in diameter at the focal point) or linear beam (about 2×5 to 1×30 mm).

It is yet another preferred embodiment of this invention includes a handheld design where the optical or electrical parts of the device may be integrated into a compact size for convenient use similar to a razor.

It is yet another preferred embodiment of this invention includes formulas for lens design including parameters for optimal focal length, laser spot size and maximal penetration depth for best clinical outcome.

It is yet another preferred embodiment of this invention includes a method of a fiber-coupled device to combine a series of diode-laser arrays into one single bundle and delivered to the treated area.

It is yet another preferred embodiment of this invention includes a method of generation a linear laser spot which can be used to scan over the treated area.

It is yet another preferred embodiment of this invention includes a method and device to combine multiple wavelength laser source having a wavelength range of 700 to 1100 nm, where at least two different wavelength laser can be integrated into one unit. The preferred laser source includes semiconductor diode-laser, most preferable at about 750, 810, 920 and 980 nm, or combination of at least two of these preferred spectra. The multiple wavelength device for hair removal is more efficient for all skin types and hair color, where various portions of the hair and surrounding tissues or blood vessels at different depth (3 to 10 mm) can be efficiently damaged.

Further preferred embodiments of the present invention will become apparent from the description of the invention which follows.

### BRIEF DESCRIPTION OF THE DRAWINGS

FIG. 1. Laser power density versus penetration depth for a focused laser beam.

FIG. 2 Schematics of system structure with the laser unit outside or inside the hand piece.

FIG. 3. Schematics of laser unit structure with focusing optics.

FIG. 4. Schematics of laser unit consisting of a diode arrays with energy Being delivered to the hand piece by fiber bundle.

FIG. 5 Schematics of lens design and hand piece configuration.

### DETAILED DESCRIPTION OF THE INVENTION AND THE PREFERRED EMBODIMENTS

The structure of hair is known as follows. It comprises of a shaft and a root which are enclosed by follicle. Located at the lower end of follicle is the papilla which is fed by blood vessels and provides nourishment to root. The main component of hair of all colors is the protein keratin. Therefore, in order to effectively and permanently prevent re-growth of hair, the papilla, blood vessels, hair shaft, or a combination of them, must be significantly damaged. Prior arts using ruby laser (at 694 nm) has good penetration due to low melanin absorption, however, it has poor blood (or oxy-hemoglobin) absorption. Prior arts using pulsed dye lasers (at 577 to 585 nm) are well absorbed by hemoglobin (Hb or HbO), but also have very high melanin absorption which prevents its energy deep into the hair papilla, which is about 3 to 10 mm, mostly 4 to 6 mm, skin depth.

Prior arts using Alexandrite (at about 755 nm) has a peak Hb absorption and relatively low (comparing to ruby laser) melanin absorption, with better penetration of about 5 mm, which is good for shallow papilla for certain kind of hair colors. The most popular laser currently used in the market is a diode laser at about 800 to 810 nm which will perform similarly to Alexandrite laser, and again, it is not effective for papilla located in a deeper range of 6 to 10 mm. Furthermore, for higher melanin absorption in darker skin and lighter hair color, these high-power (50 to 100 W) diode lasers could lead to skin damage, even a longer pulse width was proposed to reduce the peak power.

The above prior arts using alexandrite or diode laser (at about 800 nm) has higher melanin absorption than that of longer wavelength laser (900 to 980 nm) which limits the penetration depth and also causes more epidermis damage.

Prior art proposed to use a laser at about 900 to 950 nm, (U.S. Pat. No. 6,544,255) has low absorption in dermis, melanin and blood (or hemoglobin), but strong absorption of the keratin in the hair shaft. This prior art also disclosed the use of a second-harmonic wavelength at about 450 to 475 nm, which however requires an intrafollicular probe to reach a skin depth beyond the effective range (about 4 to 6 mm) of the transcutaneous method. This prior art did not specify the type of laser, which the present inventor believes that it should be a diode laser, since no other commercial lasers available at this spectral range. Therefore, the second-harmonic of a diode laser (having a peak power not higher than 2 KW) is almost impossible or has a extremely low efficient. The proposed method of dual wavelength of 900 to 950 nm and 450 to 470 nm for a dual-application is not technically practical.

This invention discloses the use of multi-wavelength (generated from one single laser unit) to overcome the above described limitations. The preferred embodiments will cover a wide range of skin depth penetration (3 to 10 mm) and cause the damage of blood vessels feeding the papilla, hair shaft, root, follicles, or combined damage of above. The deeper penetration and direct damage of the hair shaft with combined damage of blood vessels and papilla will improve the efficacy of hair removal and cover all skin types and hair colors.

Penetration Depth at Various Wavelength

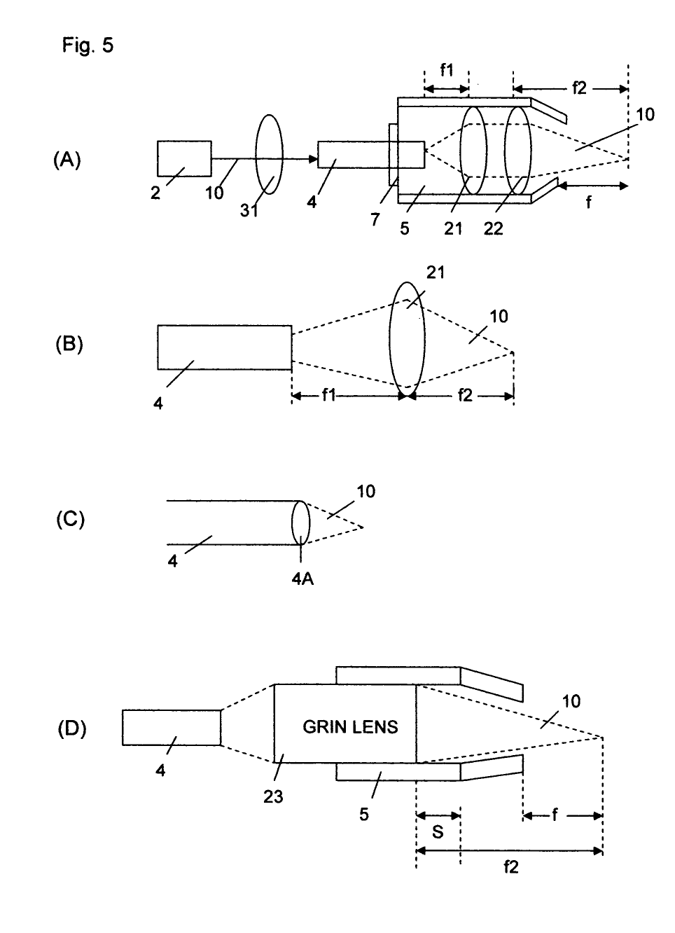

The penetration depth of the selected lasers at various wavelength (694 to 980 nm) is analyzed as follows: The absorption depth (d) and the absorption coefficient (A) define the power (or intensity I) density (P), normalized by its value on the treated skin surface of a laser when it is propagating in an absorbing medium by a revised Beer's law P=Bexp(−dA), where B is a focusing factor having a typical value of B=(1, 4, 16) at the position d=(0, 0.5, 1.0)f, f being the effective focal length of the optics (J. T. Lin, unpublished formula). For a non-focused laser case, as used in all prior arts, B=1.0 for all d. Note that f is the effective focal length inside the treated skin which is about 4 to 10 mm, in comparing to the actual focal length of the optics, about 10 to 25 mm. Greater details will be shown later in FIG. 5. Given A (in melanin) of skin epidermis about (250, 150, 105, 65, 50) (1/cm) at laser wavelength of (694, 750, 810, 920, 980) nm, respectively, we may easily calculate P after the absorption of epidermis (assumed to be about 0.2 mm, or 0.02 cm) P=(0.4, 5, 12, 27, 37)% for the above lasers for the non-focused case B=1. These calculated data give us one of the important criteria of selecting lasers for various penetration depth. We may further include the effects of water absorption in the skin (for a depth of 5 mm) given by about 1% for (694 to 800) nm, and about 10% at 920 nm, 20% at 980 nm, the remaining percentage of laser power at a skin depth about 5.2 mm, prior to the absorption of keratin or hemoglobin, is given by (0.4, 5, 12, 24, 30)% for a non-focused laser at (694, 750, 810, 920, 980) nm, respectively.

Therefore, We May Expect the Following.

Given the same treating laser power, the “available power” (AP) after the absorption of melanin and water, given by 1-exp(−dA) for non-focused case, at a skin depth d=5.2 nm, at 980 and 920 nm is about 5 to 6 times of AP at 750 nm and about 2 to 2.5 times of AP at 800 nm. In other words, lower power, 2 to 6 times less, is needed when lasers at (920 to 980) nm is used, in comparing to that of shorter wavelength of (750 to 800) nm used in prior arts. Therefore, much less damage of skin epidermis would be expected for lasers at (920 to 980) nm, while deeper penetration is also available, in comparing to that of (750 to 800) nm. However, another factor of absorption coefficient (A2) in deoxy hemoglobin must also be considered, where A2 is about (90, 40, 38, 25) (1/cm) at (750, 800, 920, 980) nm, respectively. Finally, we also need to include the absorption coefficient (A3) in oxy-hemoglobin given, respectively, by about (25, 38, 60, 65, and 68) (1/cm). The overall penetration depth and temperature rising of the treated skin (at various depth) may be calculated by the above absorption coefficients A, A2 and A3, in addition to water absorption and energy loss due to light scattering on and inside the treated skin. The above analysis provides the critical elements of the advantages of this invention and it has not been disclosed in prior arts.

Penetration Depth of Focused Beam

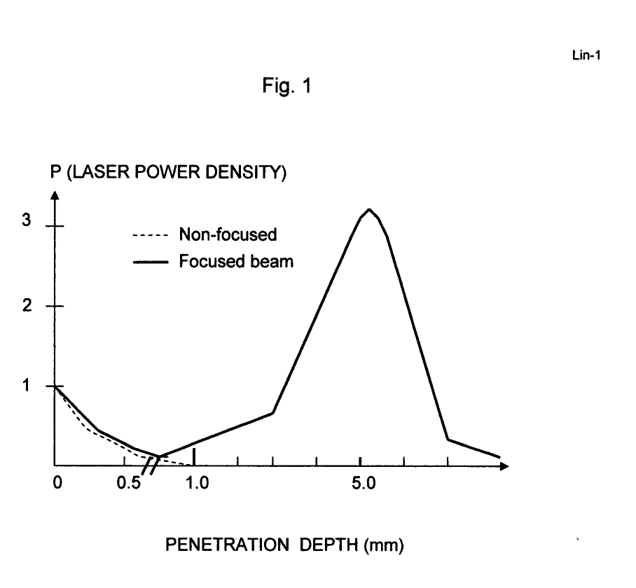

As a preferred example for a laser at 920 nm having A=65 (1/cm) and B=(1, 1.1, 1.23, 4, 16, 4) at d=(0, 0.25, 0.5, 2.5, 5.0, 7.5) mm for an effective focal length f=5 mm, one calculates P which is shown in FIG. 1, for the non-focused beam (dashed curve) and focused beam (solid curve). The important feature of FIG. 1 is the increase of penetration depth (d) from about 0.25 mm (non-focused beam) to 5 mm (focused beam), in addition to the increase of P, from 0.2 to 3.2 for d equals 0.25 to 5.0 mm.

The increase of maximal penetration depth and P in focused beam is another important elements of this invention. The P value of 3.2 at d=5.0 mm, a typical position of the papilla (assuming P=1.0 on skin surface, d=0) provides us with sufficient laser power density to cause damage of hair root, papilla and the surrounding blood vessels. Therefore the required laser power for hair removal in focused beam, is about 10 to 15 times less than that of the non-focused beam and the risk of epidermis damage is significantly reduced. In contrast, prior arts using non-focused laser having a shallow penetration depth require a much higher laser power on the skin surface in order to reach the penetration depth about 4 to 5 mm. The significant epidermis damage of prior arts requires a cooling means on the treated skin.

By simple geometry, we may derive (J. T. Lin, unpublished) B equals to the square of f/(f-d) or R1/R2, where R1 and R2 are the laser spot size on skin surface (d=0) and at the focal point f-d. The preferred parameters includes an effective focal length f=(3-10) mm, focal length of the optics F=(5-25)mm, such that R1=fRs/F=(2-5) mm, for the laser spot at the focusing optics surface of about Rs=(5-10) mm. Therefore, the preferred B value at the focal point includes B equals about 4 to 32, most preferable about 10 to 16. These preferred parameters are the key elements for the lens design and hand piece configuration (to be shown later in FIG. 5) of this invention.

The existing systems for hair removal were designed for large area treatment, therefore, a typical laser spot of about 9 mm was required for reasonable fast procedure. Another preferred embodiment of this invention is to use a small spot of about 5×5 mm or a line shape spot of 1 to 2 mm in width and about 5 to 30 mm in length. This compact device, having a laser output area about 10 to 30 mm square, will only require an output power of about 5 to 15 W which is only about 1/20 to 1/30 of the conventional system, about 50 to 100 W. This low power requirement allows us to reduce the system dimension and weight over 30 folds and make it possible to have the light source or optics integrated into a size comparable to a commercial razor. The preferred compact device is designed particularly for small area treatment, such as facial hair, and the handheld piece can be plugged into a standard AC power outlet or operated by a DC battery. Greater details are shown as follows.

Preferred Hand Piece Configurations

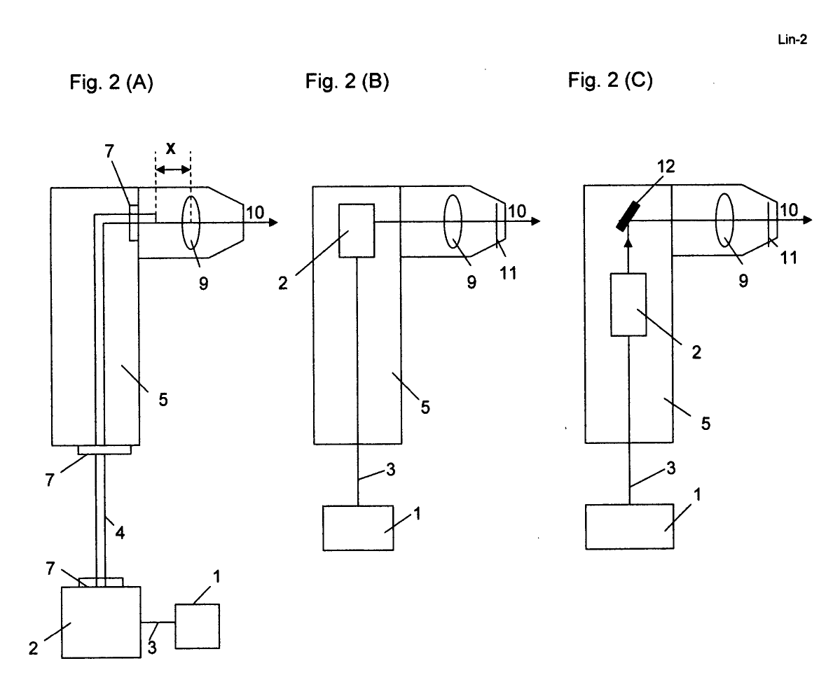

As shown in FIG. 2, the preferred embodiment of a compact device includes configuration of (A), (B) or (C), with or without an optical fiber. FIG. 2(A) shows a power supply and controller 1 connected to the laser unit 2 by a power line 3; the laser unit 2 is further coupled by an optical fiber 4 to a hand piece 5. FIG. (B) and (C) show alternatives having the laser unit 2 integrated into the hand piece 5 without the need of optical fiber. The preferred housing of the hand piece, as shown in FIG. 2 includes a dimension about 1 to 2 cm wide and thick, and about 5 to 10 cm long.

As shown by FIG. 2(A), the optical fiber 4 is connected to the hand piece housing 5 and having its output beam 10 coupled to a lens 9. The lens 9 includes a spherical, aspherical or cylinder lens, or a combination of more than one lens which controls the size and shape of the output beam 10 on the treated surface including a round spot having a diameter about 2 to 5 mm, or a line spot having a width about 1 to 3 mm and length about 5 to 30 mm. The spot size and shape of the output beam 10 may be controlled by the lens location X about 2 to 5 mm and the focal length of the lens 9 (F) about 5 to 25 mm as discussed earlier for deep penetration of about 3 to 10 mm.

As shown in FIG. 2(B), the laser unit 2 is integrated inside the hand piece 5 having an output window 11 which can be detached for cleaning and for protecting the optics 9. The window 11 shall be highly transparent to the infrared laser used in this invention ranging 0.7 to 1.1 micron. FIG. 1(C) shows another alternative where the output beam is reflected by a 45 degree angle high reflecting (HR) optics 12, HR-coated at the preferred laser IR wavelength. Example (B) and (C) are alternatives particularly for its simplicity and suitable for a low-power device, 0.5 to 5 W, for small area treatment. In FIG. 1(A) with the laser unit 2 connected to the hand piece 5 by an optical fiber, the dimension of the laser unit 2 is not limited by the space available as that of (B) or (C), therefore medium-power, 5 to 15 W, output is available with an air or water cooling. The preferred cooling means for the laser unit 2 in configuration 1 (B) or (C) includes conductive (or electronic) or miniature air cooler, whereas water or air cooler is preferred in (A).

Diode Array Configuration

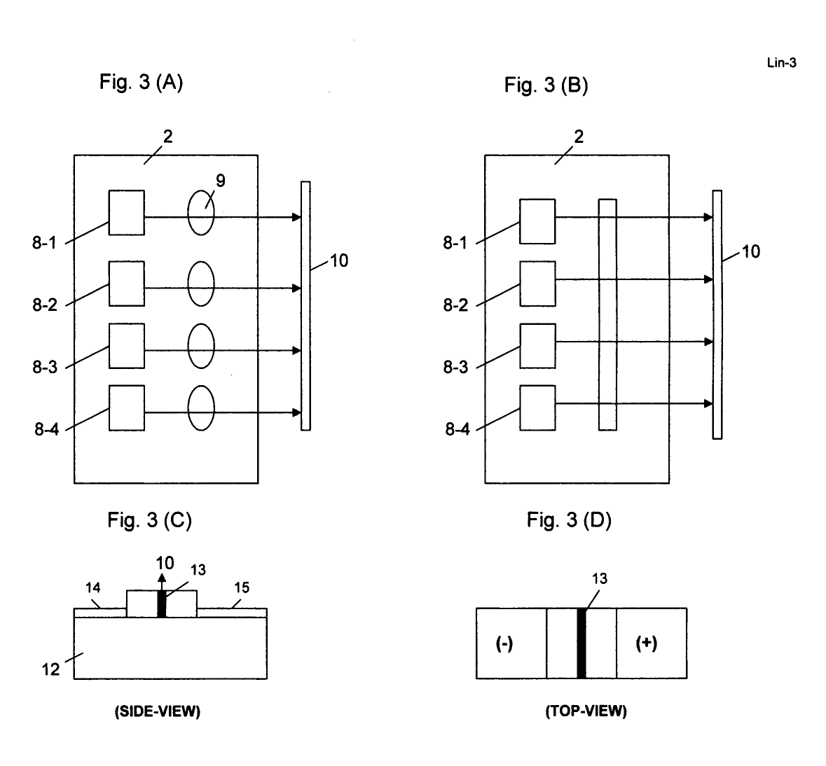

Other preferred examples of configuration of the laser unit 2 are shown in FIG. 3, where 3(A) shows a series of diode laser (chip or array) 8-1, 8-2, . . . 8-N, and each of them coupled to a lens 9 to produce a line-shape output beam 10, where the total power is given by P=NPi, with Pi being the single diode power and N is at least 1, most preferable N=2 to 5, for a line-shape output beam dimension of about 5 to 30 mm in length on the treated area. Alternative of lens 9 is a single strip lens as shown by FIG. 3(B). Configuration 3(A) and (B) are most preferable for the hand piece structure of FIG. 2(B) and 2(C) without the use of optical fiber 4. FIG. 3(C) and 3(D) show one preferred typical configuration of the diode array 8, where 12 is the heat exchange conductive plate having a dimension about 25×11×8 mm; the diode array bar 13 emitting an output light 10 having a dimensional about 11×0.1×0.1 mm and connected to the (+), (−) electrodes 14 and 15. Each array may have more than one bar having an output power about 3 to 10 W, operated in continuous wave (CW), or a quasi-CW mode having a typical peak power about 30 to 60 W in each bar.

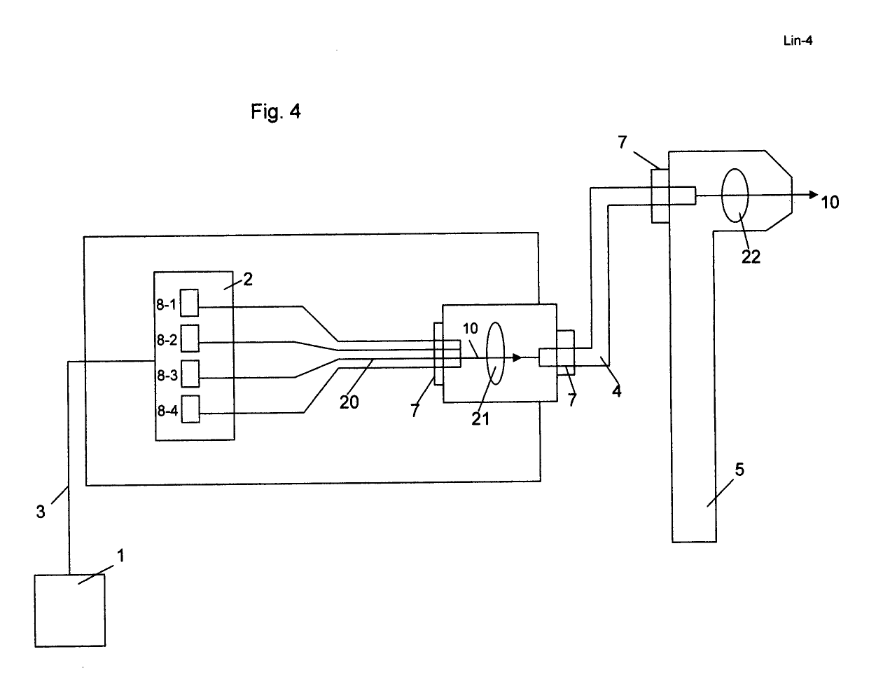

FIG. 4 shows another preferred configuration of the diode arrays, 8-1 to 8-N, combined to a fiber bundle 20 which is further coupled to a single fiber 4 by a lens 21. This configuration allows us to combine the power of a series of single diode laser (chip or array) to a standard single fiber 4 which further delivers the output beam 10 to the hand piece 5 having a focusing lens 22 which has the same function as the lens 9 in FIG. 2. The fiber bundle configuration also allows us to mix diode arrays at different wavelengths which may be further easily controlled such that they are delivered to the treated area simultaneously or sequentially, while the output beam spot and shape remain unchanged. In addition, a visible low-power aiming laser may be integrated in the fiber bundle.

The preferred embodiment of this invention includes the basic diode laser having a wavelength of about 700 to 1100 nm, and most preferable selected from one of the groups of: 700 to 760 nm, 780 to 820 nm, 900 to 930 nm and 970 to 990 nm. It further includes two or three wavelengths selected for the above described groups. For example, the laser unit 2 of FIG. 2 and FIG. 3 includes a diode laser at a single wavelength at about 750, 810, 920 or 980 nm. Another preferred example, the diode laser arrays, show in FIG. 3 and 4, could have the same wavelength at about 0.7 to 1.1 micron, or combined two or three wavelengths of about 750, 810, 920, 980 nm.

The preferred embodiment of this invention also includes that the diode arrays are packed side by side, that is the length directions (about 11 mm) of the array bars shown in FIG. 3(C) are lined up for a total bar length of about 11N.

The advantages of the above multi-wavelength configuration include the following. Two or three different wavelengths selected from short (about 700 nm) to long (about 980 nm) wavelength allow the laser energy to simultaneously or sequentially target various portion of hair structure and tissue at various depth, such that efficacy for all skin types and hair colors may be improved over prior arts using only one single wavelength. The most preferable embodiment includes a selection of two or three wavelengths from the following group which are commercially available for output power about 5 to 25 W: 750, 810, 920 and 980 nm. Some preferred examples include: (1) the laser at about 920 nm (having a maximal keratin hemoglobin) will effectively damage the hair itself (shaft and root), whereas a second wavelength at about 810 nm will effectively damage papilla by its high absorption of blood hemoglobin; (2) laser at about 750 nm (having a maximal deoxy-hemoglobin) will cause effective damage of blood vessels of papilla at a depth up to about 5 mm, whereas a second wavelength at about 920 or 980 nm will cause damage of the hair shaft or root at a deeper depth about 6 to 10 mm; and (3) a combination of 920 and 980 nm will cause damage of both the hair itself (shaft and root) and the blood feeding the papilla at a deep depth of about 4 to 10 mm and effective for all skin types and hair colors. The above specified features are not disclosed in the prior arts and provide advantages over prior arts including improved efficacy, less re-treatment frequency and suitable for all skin types and hair colors. The features are available only after the detailed analysis on the absorption coefficients of hemoglobin, keratin, melanin and tissue/water at the spectral ranges of 700 to 980 nm as shown earlier.

The pulse widths and time delays between pulses which are critical in prior arts, but not in this invention. When two or more wavelengths as proposed in this invention are irradiating on the hair-bearing skin, a fixed pulse duration about 5 msec to 100 msec would be effective in removing hair in all skin types and hair colors. This feature simplifies the system design suitable for low power, low cost, compact device.

Lens Design for Spot Size Control

The preferred configurations and lens design for optics and fiber integrated in the hand piece 5 are shown in FIG. 5(A) to 5(D). Referring to FIG. 5(A), the laser unit 2 having an output beam 10 is focused by a lens 31 into the fiber 4 which is connected by a standard SMA connector 7 to the hand piece 5. Focusing lens 21 produces a collimated beam which is then focused into the treated skin by lens 22. The effective focal length inside the skin (f) is controlled by the focal length of lens 21 and 22, f1 and f2, and the length of the holder 5 having its output end contacted to the treated surface. The preferred parameter includes f1 about 2 to 5 mm, f2 about 10 to 25 mm and f about 3 to 10 mm which covers a wide range of depth of the hair root or papilla in various skin and hair types.

FIG. 5(B) shows an alternative configuration which only consists of one focusing lens 21 having a front focal length f1 about 2 to 5 mm and a back focal length f2 about 10 to 25 mm. The focusing lens 21 and 22 in FIG. 5(A) and 5(B) include spherical, aspherical or cylinder lens being substantial transparent to the wavelength of the basic laser 2, 0.7 to 1.1 microns.

FIG. 5(C) shows another preferred embodiment of this invention in which the optical fiber 4 has a curved end face 4A such that the output beam 10 is focused into the treated skin. The end face 4A may be contacted or almost contacted to the treated skin surface and have an effective focal length about 4 to 10 mm to match the depth of hair root or papilla for various skin and hair types.

FIG. 5(D) shows a graded-index (GRIN) lens 23 is used to couple the output from the fiber 4 and produce a focusing output 10. The focal length f and f2 have the same preferred rays as that of FIG. 5(A).

The preferred laser spot size in FIG. 5 includes a diameter about 5 to 10 mm on the treated skin surface and about 2 to 5 mm at the focal point which is about 3 to 10 mm deep. The above preferred parameters are calculated based on the revised Beer's law introduced earlier to meet optimal clinical outcome including low laser power required, low risk of epidermis damage and high efficacy of hair removal. Furthermore, the preferred configurations of FIG. 5 are also the critical elements for a low-power, compact device disclosed in this invention which are not disclosed in prior arts.

Another preferred embodiment of the present invention is to use the handheld piece 5 shown in FIG. 2 manually scan over the treated area. Multiple treatments (1 to 2 times) may be required by the teaching of this invention, less than the existing systems (about 3 to 4 times). The typical energy per pulse of existing system with a large beam (about 9 mm spot) is about 20 J. A much low energy per pulse of about 0.5 to 5 J would be needed using a focused beam proposed in this invention, since the energy/pulse required is proportional to the area of the laser spot. To minimize skin surface damage in dark skin, a longer pulse is normally required for a laser having a wavelength shorter than about 850 nm, whereas a fixed pulse duration may be used in a laser having a wavelength at about 920 to 950 nm, which is absorbed mainly by the keratin component of the hair in all types. In addition, as discussed earlier, due to the lower absorption in melanin and the focused laser spot, much lower power (about 10 to 15 times less) is needed for laser at 920 to 950 nm, comparing to that of non-focused laser at 750 to 800 nm to reach the same skin depth. Lower power laser also has the advantage of less epidermis damage.

The above desired features and advantages of method and device disclosed in the present invention are based on the laser interaction with various portions of the hair and blood vessels, the new feature of penetration depth in a focused beam and absorption of skin and hair at various laser wavelength. These advantages are not available by prior arts having a single wavelength and operated at a non-focused mode. The compact novel design is also achievable only under the teaching of this invention, and not by that of prior arts or the existing commercial systems.

While the invention has been shown and described with reference to the preferred embodiments thereof, it will be understood by those skilled in the art that the foregoing and other changes and variations in form and detail may be made therein without departing from the spirit, scope and teaching of the invention. Accordingly, threshold and apparatus herein disclosed are to be considered merely as illustrative and the invention is to be limited only as set forth in the claims.

I claim:

1. A method of hair removal comprising the steps of:
(a) selecting a laser beam having a predetermined energy, spot size and wavelength; and
(b) selecting a beam delivery means which delivers said laser beam energy to a targeted area,
whereby one or more than one of said targeted area is damaged to prevent the re-growth of hair.

2. A method of claim 1, wherein said laser beam includes infrared semiconductor diode laser having a wavelength of about 0.7 to 1.1 microns, energy per pulse of about 0.5 to 5.0 J and power of about 0.5 to 15 W and a spot size of about 5 to 10 mm on the skin surface which is focused to a spot size of about 2 to 5 mm by a focusing lens having a focal length about 5 to 25 mm.

3. A method of claim 1, wherein said targeted area includes the hair shaft, root, hair follicle, blood vessels feeding the papilla, or blood vessels in the papilla.

4. A method of claim 1, wherein said delivery means includes an optical fiber or fiber bundle which delivers said laser beam having one or more than one wavelength to said targeted area.

5. A method of claim 4, wherein said optical fibers is further connected to a hand piece containing the laser unit and focusing optics including spherical, aspherical, cylinder or graded-index (GRIN) lens.

6. The method of claim 1, wherein said laser beam is generated from a laser unit consisting of about 1 to 5 diode arrays having a wavelength about 0.7 to 1.1 microns.

7. The method of claim 6, wherein said diode array includes diode laser emitting the same wavelength at about 0.7 to 1.1 microns, or a combination of 2 to 3 different wavelengths selected from the ranges of about 700 to 760 nm, 780 to 820 nm, 900 to 930 nm, or 970 to 990 nm, and most preferable at about 750, 810, 920, or 980 nm.

8. The method of claim 6, wherein said diode array includes one or more than one emitting bar attached to a heat exchanger and having a length about 11 mm, output average power of about 3 to 10 W operated at continuous wave or quasi-continuous wave having a peak power about 30 to 60 W in each bar.

9. The surgical method of claim 6, wherein said diode array output beams are coupled to a lens or a set of lens to produce a round beam spot about 2 to 5 mm in diameter, or a line spot about 1 to 3 mm wide and 5 to 30 mm long, at the treated skin surface.

10. A method of claim 1, wherein said laser beam having one or more than one wavelength is focused and delivered to various said targeted area at a penetration depth of about 3 to 10 mm to cause the damage of one or more than one of said targeted area, where the penetration depth (d) is defined by a revised Beer's law P=Bexp(−dA), P is the laser power density, A is the absorption coefficient, and B is a focusing factor having a value about 4 to 32, most preferable about 10 to 16 at the focal point.

11. A system of laser hair removal consisting of:
(a) a laser beam having a predetermined energy, spot size, pulse width and wavelength; and
(b) a beam delivery means to deliver said laser beam energy to a targeted area, whereby said targeted area is damaged to prevent the re-growth of hair.

12. A system of claim 11, wherein said laser beam includes diode laser having a wavelength of about 0.7 to 1.1 microns, energy per pulse of about 0.5 to 5.0 J and power of about 0.5 to 15 W and a spot size of about 5 to 10 mm on the skin surface which is focused to a spot size of about 2 to 5 mm by a focusing lens having a focal length about 5 to 25 mm.

13. A system of claim 11, wherein said targeted area includes one or more than one of the following targets: the hair shaft and root, hair follicle, blood vessels feeding the papilla, or blood vessels in the papilla.

14. A system of claim 11, wherein said delivery means includes an optical fiber or fiber bundle which delivers said laser beam having one or more than one wavelength to said targeted area.

15. A system of claim 11, wherein said optical fiber is further connected to a hand piece containing the laser unit and focusing optics including spherical, aspherical, cylinder or graded-index (GRIN) lens.

16. A system of claim 11, wherein said laser beam is generated from a laser unit consisting of about 1 to 5 diode arrays having a wavelength about 0.7 to 1.1 microns, or a combination of 2 to 3 different wavelengths selected from the ranges of about 690 to 720 nm, 780 to 820 nm, 900 to 930 nm or 970 to 990 nm, and 700 to 760 nm, 780 to 820 nm, 900 to 930 nm, or 970 to 990 nm, and most preferable at about 750, 810, 920, or 980 nm.

17. A system of claim 16, wherein said diode array includes one or more than one emitting bar attached to a heat exchanger and having a length about 11 mm, output average power of about 3 to 10 W operated at continuous wave or quasi-continuous wave having a peak power about 30 to 60 W in each bar.

18. A system of claim 16, wherein said diode array output beams are coupled to a lens or a set of lens which produces a round beam spot about 2 to 5 mm in diameter, or a line spot of about 2 to 5 mm wide and 5 to 30 mm long at the treated skin surface.

19. A system of claim 11, wherein said laser beam having one or more than one wavelength is focused and delivered to various said targeted area at a penetration depth (d) of about 3 to 10 mm to cause the damage of one or more than one of said targeted area, where the penetration depth (d) is defined by a revised Beer's law P=Bexp(−dA), P is the laser power density, A is the absorption coefficient, and B is a focusing factor having a value about 4 to 32, most preferable about 10 to 16 at the focal point.

20. A system of claim 15, wherein said hand piece includes a compact dimension about 1 to 2 cm in width and thickness, and about 5 to 10 cm in length.
